(19) United States
(12) Patent Application Publication
(10) Pub. No.: US 2011/0151461 A1
(43) Pub. Date: Jun. 23, 2011

(54) DETECTION ALGORITHM FOR PCR ASSAY

(75) Inventors: John Robert Link, Wilmington, DE (US); Barry Edward Boyes, Wilmington, DE (US)

(73) Assignee: Smiths Detection-Edgewood Inc., Edgewood, MD (US)

(21) Appl. No.: 13/057,735

(22) PCT Filed: Aug. 10, 2009
(86) PCT No.: PCT/US09/53253

§ 371 Date: Feb. 4, 2011

Related U.S. Application Data
(60) Provisional application No. 61/136,040, filed on Aug. 8, 2008.

(51) Int. Cl. C12Q 1/68

(52) U.S. Cl. 435/6.11

(57) ABSTRACT

The application provides methods for improving the detection accuracy of the binding of labeled nucleic acid probes, such as those used in PCR reactions. One such method comprises measuring the label intensity, e.g. fluorescence, at two different temperatures, a higher temperature and a lower temperature, and then calculating the ratio of the label intensity at the lower temperature over the label intensity at the higher temperature. Another method comprises measuring the label intensity at least two points in time post-PCR and calculating the slope of the label intensity as a function of time. Measuring the hybridization kinetics of the probe binding to the target nucleic acid allows an on-rate slope to be calculated which gives this method good specificity of detection.

This application claims priority under 35 U.S.C. §119(e) to U.S. provisional application No. 61/136,040, filed 8 Aug. 2008, which is hereby incorporated by reference in its entirety.

### BACKGROUND

The present application relates generally to the field of detection of nucleic acids using polymerase chain reaction (PCR). The application provides methods for increasing specificity, and therefore sensitivity, of the detection of target nucleic acids using PCR by calculating the effect of temperature on the hybridization of a labeled probe to the target, and by calculating the hybridization rate of the probe to the target as a function of label intensity as a function of time.

The amplification of nucleic acids has been an invaluable tool for the detection of specific nucleic acids in a sample. PCR is used to amplify the nucleic acid using thermocycling of a heat stable DNA polymerase, such as Taq polymerase, in a reaction comprising the target nucleic acid, primers for DNA polymerization that are complementary to the target nucleic acid, as well as the necessary nucleosides and buffer reagents, as described, for instance, in U.S. Pat. No. 4,683,202. Many variations of PCR have been developed for specific needs, such as can be found in Current Protocols in Molecular Biology (April 2008, Print ISSN: 1934-3639; Online ISSN: 1934-3647). It is a widely used technique for detecting the presence of DNA and RNA targets for a variety of purposes, including, for example, pathogen detection ex vivo and from environmental samples, in vitro diagnostics, genetic analyses, forensics, food and agricultural testing, and parentage testing.

PCR has evolved from technique performed only under controlled laboratory conditions to a technique useful for field testing. This evolution has been made possible by the advent of handheld PCR devices. Such devices may be particularly suited for the detection of pathogens, forensic sampling, or even rapid diagnosis of medical conditions without the need for expensive, time consuming and laborious laboratory processing. The use of PCR in the field is particularly important to counter bioterrorism, because rapid and accurate identification of bioweapons is crucial.

Field applications still require a robust and accurate assay, despite the lack of a controlled laboratory environment. While field assays are about on par with laboratory assays in detection of target DNA, comparison of PCR assays versus traditional culture techniques for the diagnosis of bacterial infection from tissue samples indicated the PCR methods were less sensitive than a 72 hour laboratory culture (Emanuel et al. J. Clin. Microbiol. (2002) 41:689-693). Thus, improved PCR sensitivity is a goal for developing rapid detection methods.

Signal specificity remains a major limitation of the sensitivity of any PCR assay, particularly when using fluorescent reporter molecules for measuring the reaction kinetics and amount of amplified target in the reaction. While subtraction of the background fluorescence in a negative control reaction is generally used to account for non-specific hybridization, this method remains crude, at best, limiting the lower threshold amount at which a target nucleic must be present in order to be detected.

### SUMMARY OF THE INVENTION

Provided herein is a method for detecting hybridization of a labeled nucleic acid probe to its complementry nucleic acid target comprising (a) contacting a sample suspected of containing a target nucleic acid with a labeled nucleic acid probe that hybridizes with the target nucleic acid; (b) measuring the label intensity at a first temperature and at a second temperature, wherein the first temperature is lower than the second temperature; (c) calculating the ratio of (i) the label intensity at the first temperature to (i) the label intensity at the second temperature, wherein a ratio of at least 0.8 indicates the presence of the target nucleic acid. In a further embodiment, steps (b) and (c) are repeated at least twice. In a further embodiment, steps (b) and (c) are repeated at the same first and second temperatures. The measurement of the first temperature can occur before the measurement of the second temperature or vice versa. In a further embodiment, the method is used as a post PCR detection technique. In a further embodiment, the method further comprises measuring the label intensity at single temperature at 3 or more points in time, for instance 15, 30, and 45 seconds, after the sample is brought to the said temperature. In a further embodiment, the method further comprises measuring the label intensity at different temperatures at three or more points in time, such as at 15, 30, and 45 seconds.

In one embodiment, a ratio of at least 0.9 indicates the presence of the target nucleic acid.

In another embodiment, the first temperature is below the Tm of the labeled nucleic acid probe and the second temperature is above the Tm of the labeled nucleic acid probe. In a further embodiment, the first temperature is about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C. or about 72° C.

In another embodiment, the second temperature is about 85° C., about 86° C., about 87° C., about 88° C., about 89° C., about 90° C., about 91° C., about 92° C., about 93° C., about 94° C., or about 95° C.

In another embodiment, the second temperature is about 85° C., about 86° C., about 87° C., about 88° C., about 89° C., about 90° C., about 91° C., about 92° C., about 93° C., about 94° C., or about 95° C.

In another embodiment, the first temperature is about 50° C. and the second temperature is about 95° C.

In another embodiment, the labeled nucleic acid probe comprises a fluorescent label. In a further embodiment, the nucleic acid probe further comprises a quencher molecule that absorbs the emission of the fluorescent label such that when the quencher molecule and fluorescent label are in relatively close proximity, the fluorescent emission of the fluorescent label is undetectable or at least less detectable than when the quencher molecule and fluorescent label are not in close proximity. In a further embodiment, the nucleic acid probe is a molecular beacon or a linear probe.

In one embodiment, the target nucleic acid is DNA or RNA.

In one embodiment, an average ratio is calculated based on repeated measurements.

In a further embodiment, the measuring is done following a PCR reaction.

In another embodiment, the PCR reaction comprises i) contacting a sample suspected of containing the target nucleic acid with the labeled nucleic acid probe in a solution comprising suitable primers, enzymes and substrates to form a reaction mixture; and ii) cycling said reaction mixture at denaturing, annealing and extension temperatures suitable for amplification of the target nucleic acid.

Further provided herein is a method for detecting hybridization of a labeled nucleic acid probe to its target nucleic acid comprising (a) contacting a sample suspected of containing a target nucleic acid with a labeled nucleic acid probe that hybridizes with the target nucleic acid; (b) measuring the label intensity at least two different points in time; and (c) calculating the slope of the label intensity as a function of time. The measuring at different points in time, as done in step (b), can be done under isothermic conditions or at different temperatures.

In a further embodiment, a positive slope indicates the presence of the target nucleic acid when the intensity of the signal generated by the labeled nucleic acid probe increases over time when bound to the target nucleic acid as compared to the intensity of the signal generated by the labeled nucleic acid probe when it is not bound to the target nucleic acid. In a further embodiment, a negative or zero slope indicates the absence of the target nucleic acid

In a further embodiment, the nucleic acid probe is a molecular beacon or a linear probe.

In one embodiment, the target nucleic acid is DNA or RNA.

In one embodiment, the measuring is done during isothermal conditions. In a further embodiment, the measuring is done following a PCR reaction. In a further embodiment, the measuring is completed within the time period of about 1 to about 10 minutes following the completion of the PCR reaction. In a further embodiment, the measuring is done at least five different points in time. In a further embodiment, the measuring is done at 15, 30, and 45 seconds following the completion of PCR.

In another embodiment, the PCR reaction comprises (i) contacting a sample suspected of containing the target nucleic acid with the labeled nucleic acid probe in a solution comprising suitable primers, enzymes, substrates and buffer to form a reaction mixture; and (ii) cycling said reaction mixture at denaturing, annealing and extension temperatures suitable for amplification of the target nucleic acid. In another embodiment, the annealing and extension temperatures are the same.

In one embodiment, the slope is calculated by taking the first derivative of the label intensity as a function of time. In a further embodiment, the label intensity as a function of time is calculated by least squares fitting the label intensity measurements as a function of time. In a further embodiment, the slope is calculated by fitting the label intensity data as a function of time to the following equation: y=mx+b, wherein y is label intensity, x is time, and m is the slope.

In a further embodiment, the hybridization kinetics of the labeled nucleic acid probe and target nucleic acid based on the slope of label intensity is calculated as a function of time.

### BRIEF DESCRIPTION OF THE DRAWINGS

FIG. 1 is a graph showing the ratio of the fluorescent values below the beacon Tm divided by the fluorescence values above the beacon Tm. Positive ratios (+) were found only for samples containing bacillus globigii (BG) template, which is a surrogate organism for studying biological weapons, and negative ratios (−) were found for control samples that lacked BG template. Thus, FIG. 1 shows that the use of ratio values can be used to positively detect even small quantities of target sample while minimizing false positives.

FIG. 2 shows the results of measuring the slope of the fluorescence values over 3-10 minutes of six samples. Only samples containing BG templates, the target sequence, showed positive slopes.

FIG. 3 shows the results of PCR reactions performed to determine whether label intensity at two temperatures at two different times can be used to accurately detect the presence of a target nucleic acid, a nucleic acid specific for anthrax, while minimizing false positives. The results show that anthrax was reliably detected.

FIG. 4 shows the results of PCR reactions performed to determine whether label intensity at two temperatures at two different times can be used to accurately detect the presence of a target nucleic acid, a nucleic acid specific for tularemia, while minimizing false positives. The results show that tularemia was reliably detected.

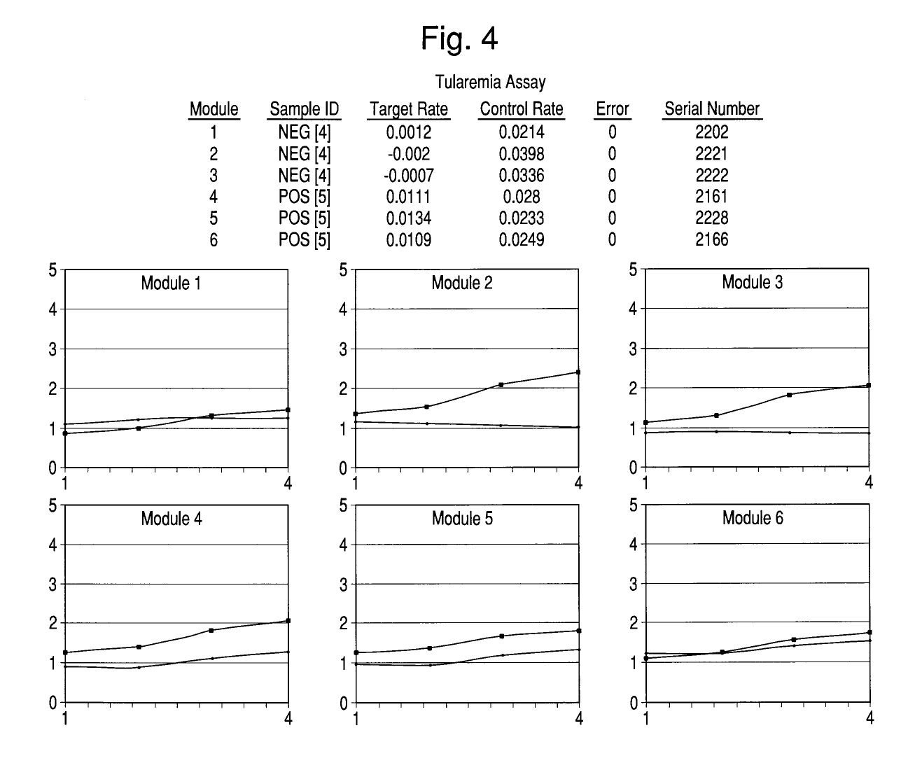

### DETAILED DESCRIPTION OF THE PREFERRED EMBODIMENTS

By “nucleic acid probe,” it is meant an oligonucleotide which is RNA or DNA that is complementary to the target sequence and thus hybridizes specifically to the target sequence. The probe can be any suitable length, and in some embodiments, the probe is from 20 to 1000 bases long. Optionally, the probe is labeled, for example linked to at least one detectable reporter molecule. A fluorescent reporter molecule can be used as the reporter molecule. The fluorescent reporters may be attached to one end of the oligonucleotide and a fluorescent quencher molecule to the opposite end of the oligonucleotide such that when the reporter is in close proximity to the quencher, the fluorescent emission from the reporter is at least partially absorbed by the quencher, thus decreasing the detectable signal of the reporter. These molecules could be also be attached to the internal portion of the oligonucleotide. The mechanism of this quenching is known as fluorescent resonance energy transfer (FRET) and results in the probe having a higher label intensity when bound to the target versus when the probe is unbound. Other methods for labeling the probe include linking radioisotopes, single fluorophores, DNA intercalating dyes (such as SYBR Green), chemiluminescent molecules, affinity tags, and the like. Hybridization of the probe to the target can be calculated using methods and equations known in the art, such at those described in Tsourkas et al. Nuc. Acids Res. (2003) 31:1319-1330, which is hereby incorporated by reference in its entirety.

By “molecular beacon,” it is meant a probe that has sequence complementary to the target in the middle of the probe, with a heterologous sequence at the 5′ and 3′ ends, which forms a stem-loop structure when the probe is not bound to the target and is at a temperature below the effective T_{M} of the stem structure. The complementary sequence of the molecular probe may be at least 85%, at least 90%, at least 95%, at least 97%, at least 98%, or at least 99% complementary to the target. The stem-loop brings the 5′ and 3′ ends in close proximity, allowing the reporter and quencher molecules to interact, resulting in a reduced label intensity. Upon binding the target, the reporter and quencher molecules become more distant, allowing for an increased label intensity. Thus, free molecular beacons generate little or no signal, while molecular beacons that are bound to the target sequence have a much greater label intensity at temperatures near or below the effective T_{M} of the beacon-target hybrid. Molecular beacon probes are well-known in the art and are described in Maras et al., Clinica Chemica Acta (2006) 363:48-60, for example.

By “linear probe,” it is meant a probe that has no particular secondary structure. The probe may be 100% complementary to the target or only partially complementary to the target. For example, the probe may be at least 85%, at least 90%, at least 95%, at least 97%, at least 98%, or at least 99% complementary to the target.

Unless otherwise specified, “probe” refers generally to a nucleic acid probe, a molecular beacon, and a linear probe. In other words, “probe,” as used herein, encompasses a nucleic acid probe, a molecular beacon, and a linear probe unless otherwise specified.

By “target nucleic acid,” it is meant the nucleic acid in a sample to be detected using the complementary PCR primers and probes. The target may be DNA, such as genomic DNA, bacterial DNA, viral DNA, episomal DNA, or synthetic DNA. The target may be RNA, such as mRNA, rRNA, tRNA, viral RNA, bacterial RNA, or synthetic RNA. Samples containing the target nucleic acid may be from any source. Such samples include biological samples, environmental samples, clinical samples, in vitro samples, and tissue samples, for example. For example, the samples can come from a material suspected of being contaminated with a biowarfare agent. Specific examples of target nucleic acid include, but are not limited to, nucleic acids encoding at least a portion of the anthrax, tularemia, plague, and pan orthopox genomes. Methods for extracting the nucleic acid from such samples for use in PCR reactions are well known in the art and may be used.

Probes and primers may be multiplexed to detect more than one target in a single reaction. In general, different sets of primers will amplify different target nucleic acids, and the probes that detect the targets may have different labels, such as different fluorophores, such that the two targets may be distinguishable in the same reaction. Such methods are well known in the art, such as those described in Belanger et al. J. Clin. Microbiol. (2002) 40:1436-1440. In another embodiment, different sets of primers will amplify different target nucleic acids, and the probes that detect the targets can have the same labels, such as identical fluorophores. The probes can hybridize at different temperatures, which allows each probe to be distinguishable in the same reaction despite the same label. This can be thought of as multiplexing in temperature space.

Additionally, multiple reactions can be used to screen for multiple targets, such as a screen for pathogenic organisms in a sample or disease genes. Such reactions may be performed in a multiwell format, for example a 96-well plate.

By “label intensity,” it is meant the amplitude of the signal detected from the probe. The particular signal detected will depend on the probe. For example, fluorescence will be detected for a probe labeled with a fluorescent probe. When using FRET-based probe labeling, label intensity is proportional to the amount of bound probe to the target and, therefore, proportional to the amount of target in the sample. Likewise, when a DNA intercalating reporter molecule is used, such as SYBR Green, fluorescence increases as more reporter is incorporated in the newly-synthesized double stranded DNA. Therefore, the label intensity can be used as a means to determine whether the target nucleic acid is present in the sample, and optionally, the amount of target present in the sample. For instance, the label intensity can be compared to a standard curve generated using known amounts of the target, interpolating the results, and determining the amount of target in the sample. Such methods are well known in the art.

In some embodiments, a proxy unit will be used to record or represent label intensity. For example, a device may detect or record the fluorescence from a fluorescent probe as a particular voltage. This proxy unit, voltage in this specific example, can be considered the label intensity.

By “hybridize,” it is meant when two single stranded polynucleotides combine to form one strand of double stranded polynucleotide. The nucleic acids may be DNA or RNA, and may form DNA-DNA, RNA-RNA, or DNA-RNA double stranded polynucleotides or three standed hybrids. Hybridization is sequence specific, and the kinetics of hybridization can be calculated using a second order equation, as described by Tsourkas et al. Nuc. Acids Res. (2003) 31:1319-1330, for example, which is hereby incorporated by reference. The kinetics of hybridization can also be calculated by measuring the fluorescent increase of a molecular beacon over time during its hybridization to a homologous sequence. A molecular beacon is labeled on one end with a fluorescent molecule and on the other with a quencher molecule. As the molecular beacon hybridizes to its complementary sequence, the reporter fluor is physically separated from the quencher molecule and the fluorescence increases. The kinetics of hybridization can then be measured as the rate of fluorescent increase using the slope of the label intensity.

By “Tm” or “melting temperature,” it is meant the temperature at which 50% of the probe molecules are hybridized to target nucleic acids, while 50% of the probe molecules remain free in solution. The Tm can be calculated, for example, using the formula Tm=2[A+T]+4[G+C], or determined by software programs developed specifically for this purpose. Calculation of Tm is well known within the art.

By “slope of the label intensity as a function of time” it is meant the slope of a line that is a plot of the label intensity or change in label intensity as a function of time. As the probe hybridizes to its specific complementary template, the label intensity increases. Over a given time period more probe hybridizes to the template, which results in an increase of fluorescence (label intensity) over time. Thus, the slope may be measured as intensity over time or change in intensity over time. Such slopes can be calculated, for example by taking the first derivative of the label intensity as a function of time, by calculating the least squares fitting the label intensity measurements as a function of time, or by fitting the label intensity data as a function of time to the following equation: y=mx+b, where y is label intensity, x is time, and m is the slope. Any suitable method known in the art can be used to determine the slope. Further the hybridization kinetics of the labeled nucleic acid probe and target nucleic acid may be calculated based on the slope of label intensity as a function of time in this manner.

The slope of a line defined by points (x₁, y₁) and (x₂, y₂) can be determined using the following formula:

$$m = \frac{{y}_{2} - {y}_{1}}{{x}_{2} - {x}_{1}}$$

where m is the slope of the line and x₁≠x₂. This equation can be employed any number of ways by one of skill in the field. For example, a line can be fit to data and then the equation can be applied to determine the slope m. Alternatively, the slope m can be calculated for a number of different points and the values averaged to determine a slope. In some embodiments, the slope will be calculated repeatedly using data points sequential in time.

By “about,” it is meant a value that is the indicated value, plus or minus five percent of that value.

By “PCR” it is meant a repetitive target nucleic acid amplification reaction based on serial cycling of the temperature of a reaction comprising the target, which may be from a sample; a DNA polymerase such as Taq polymerase or other heat stable polymerase; at least one primer complementary to the target; a probe complementary to the amplified portion of the target; deoxynucleoside triphosphates; and a buffer solution comprising divalent cations. Suitable concentrations for the components are well known in the art and may be adjusted according to known optimization parameters. Each cycle typically includes three steps: denaturation at 85-100° C., annealing at 37-60° C., and elongation at 40-75° C. Each step may be 10-300 seconds long, preferably 30-120 seconds. The exact temperature of the steps may be varied according to the sequence of the target according to well-known parameters. For instance, the annealing temperature may be a temperature approximately 5° C. lower than the Tm of the primers, which can be calculated as described above. There are many software programs available for calculating the optimal temperatures of the PCR steps. Each cycle can comprise two steps: the first step being denaturation and the second step combining the annealing and elongation steps together. An optional initial denaturation step may be included for “hot start” polymerases. Further, a final elongation step may also be included to ensure any remaining single stranded DNA molecule is fully extended. Specific protocols for performing PCR are well known in the art and may be found, for example, in Current Protocols in Molecular Biology (April 2008, Print ISSN: 1934-3639; Online ISSN: 1934-3647).

The PCR reaction cycles can be characterized as being early, late and final stages. In the early stage, exponential amplification occurs as near 100% efficiency of doubling the target sequence occurs. As reagents are exhausted, the reaction enters the late stage and efficiency drops off. This stage is sometimes called the linear stage, though the reaction actually has a high degree of variability at this stage, depending on the availability of the reagents and polymerase performance. The final stage is a plateau at which no further target sequence accumulates due to exhaustion of the reagents and enzyme.

“Real time PCR,” also referred to as quantitative or Q-PCR and originally described as the 5′ nuclease PCR assay, refers to measuring signals generated by the enzymatic cleavage of a dual labeled probe (label intensity) during the PCR reaction. The signal generated by the cleavage of the probe which is bound to the target sequence is proportional to the amount of target sequence in the reaction. By comparing the signal to known standards, the amount of target present in the sample may be determined.

“Endpoint PCR” typically refers to measuring the label intensity in the late or final stage of the reaction. Complete exhaustion of the reagents is not required. While the target in the sample may be less precisely quantified using this method as compared to real time PCR, the target has been maximally amplified, allowing for robust detection of its present due to its greater abundance in the final stages of the PCR reaction.

“Assymetric PCR” refers to a PCR technique used to preferentially amplify one strand of the target nucleic acid more than the other. Generally, preferential amplification of the target nucleic acid is accomplished by using a large excess of the primer for the preferred nucleic acid. Specifically asymmetric PCT protocols are well known in the art.

Specific detection of the amplified target remains a limiting characteristic for all PCR reactions regardless of type or time of measuring the label intensity.

PCR can be performed in any suitable thermocycler and format, such as 96 or 384 well plates. Alternatively, the PCR reaction may be performed in a field-suitable device, such as a BIOSEEQ™ PLUS device available from Smiths Detection.

The slope of the label intensity as a function of time can be calculated automatically using computer software or hardware, for example. In some embodiments, label intensities are detected, and the output of this detection is transmitted to a processor for data manipulation. The processor performs the necessary calculations and returns the data in numerical, graphical or other interpretable output to the user. In other embodiments, the user obtains the signal intensity data and manipulates the data manually. The data can be manually manipulated using standard mathematical methods or computer programs, such as Microsoft's EXCEL program, including the 2007 version.

In one embodiment, the hybridization of a labeled nucleic acid probe to its target nucleic acid is detected by measuring the label intensity at least two different temperatures. In one embodiment, the method comprises contacting a sample suspected of containing a target nucleic acid with a labeled nucleic acid probe that hybridizes with the target nucleic acid and measuring the label intensity at a first temperature and at a second temperature, wherein the first temperature is less than the second temperature or reversed.

The ratio is calculated as the label intensity at the lower temperature divided by the label intensity at the higher temperature. Thus, the higher the resulting number, the higher the concentration of target contained in the reaction. The specific value of the ratio number depends on a number of factors including the label intensity scale of the instrument being used. A ratio of greater than one can be indicative of the presence of the target. In some embodiments, a ratio of about 0.9, 1.0, 1.1, 1.2, 1.5, 2, 5, 10, 20, or 50 indicates the presence of a target nucleic acid. The ratio can also be used to calculate the amount of target present. Such a determination can be made by comparing the ratios to standards, for example. Another aspect in determining the ratio indicating the presence of a target nucleic acid is to determine the baseline ratio of reactions which contain no target nucleic acids which could be called the noise of the assay. A ratio determining the presence of target nucleic acids could be 5 or more standard deviations above the noise, for example. In some embodiments, a ratio indicating the presence of target nucleic acids could be 2, 4, 6, 8, 10, or 15 or more standard deviations above the noise. Depending on the probe used, the amount may also be calculated directly from the ratio without need for a comparison to standards.

The temperatures at which the label intensity are measured can vary depending on the particular application, and suitable temperatures can be readily calculated by one of skill in the art. The selection of temperatures will depend on the Tm and sequence of the probe. Specifically, the higher temperature will be greater than Tm of the probe, and the lower temperature will be less than the Tm of the probe. In some embodiments, the higher temperature will be at least about 90° C. and the lower temperature will be about 50° C. or less. For example, the higher temperature can be at least 95° C., 100° C., 105° C., or 110° C., and the lower temperature can be 5° C., 10° C., 15° C., 20° C., 25° C., 30° C., 35° C., 40° C., or 45° C.

In some embodiments, intensities will be measured at more than two temperatures. For example, intensity measurements can be taken at a first, second, and third temperatures, wherein each of the first, second, and third temperatures are different. Similar values at all temperatures are indicative of specific binding. Similar values can mean values that differ by no more than 20%, no more than 15%, no more than 10%, no more than 5%, no more than 3%, or no more than 1%. By increasing the number of temperatures used, the confidence in the results can be increased.

The ratio can be calculated at one or several points in time. For example, label intensity can be measured following a PCR reaction to determine the presence of a target nucleic acid that is the subject of the PCR reaction. The label intensity can also be measured before a PCR reaction is performed. A ratio indicative of the presence of the target nucleic acid may therefore allow unnecessary PCR to be avoided.

The ratio can be calculated based on a single reading at each of the higher and lower temperatures. In the alternative, multiple readings can be taken at the lower or higher temperature and the values averaged. Such averaging can be used to prevent errors caused by incorrect readings.

In one embodiment, the hybridization of a labeled nucleic acid probe to its target nucleic acid can be detected by measuring the slope of label intensity as a function of time. Because the amount of target in samples containing target increases over time due to PCR, more probe hybridizes to the target over time. Thus, label intensity as a function of time can be used to determine the presence of target in a sample. Moreover, the label intensity as a function of time can be used to determine the hybridization kinetics of the probe/target interaction.

In one embodiment, the method comprises contacting a sample suspected of containing a target nucleic acid with a labeled nucleic acid probe that hybridizes with the target nucleic acid and measuring the label intensity at different points in time. The slope of the label intensity of a function of time is indicative of both the presence of the target nucleic acid and the kinetics of the hybridization of the probe to the target nucleic acid. Specifically, a positive slope indicates the presence of the target nucleic acid when the label intensity of the probe increases when hybridized to the target, and a negative or zero slope indicates the absence of target nucleic acid when the label intensity of the probe decreases when there is no target for the probe to hybridize with. Similarly, the slope also can be used to determine the kinetics of hybridization using well-known kinetic equations.

The slope of the label intensity as a function of time can be calculated in any number of ways readily known to one of skill in the art. For example, the label intensity data can be fitted with a line using known numerical methods. These methods include least squares fitting (both linear and non-linear), linear regression (y=mx+b), best fit exponential curve, quadratic regression, cubic regression, and polynomial regression. The slope of the label intensity can be calculated, either analytically or numerically, by finding the first derivative of the line fit to the data. Such mathematical computations are well-known to those of skill in the art.

The label intensity can be measured under isothermal conditions or at different temperatures. In one embodiment, all label intensity measurements are taken at a single temperature. In another embodiment, the label intensity measurements can be taken at different temperatures. This situation may occur during the cooling or heating of a PCR reaction mixture. In yet another example, the label intensity can be measures at different temperatures. Based on this data, different slopes can be calculated. For example, multiple data points can be taken at the first temperature, T1, followed by taking multiple data points at the second temperature, T2. Based on this data, two different slopes can be calculated, one corresponding to the T1 data and one corresponding to the T2 data. By varying the temperature at which label intensity is measured, the kinetics of hybridization can be studied.

The intensity measurements can be taken at any time. The measurements can be taken following PCR or taken during PCR. In some embodiments, the measurements are taken in the ten minutes following PCR. The measurements can also be taken in five, four, three, two, or one minutes following PCR. In one embodiment, measurements are taken at about 15 sec., 30 sec., and 45 sec. The time between measurements can vary. For example, the measurements can be separated by at least about 10 sec., about 15 sec., or about 20 sec. The measurements can also be taken in rapid succession over some period of time.

The number of intensity measurements can also vary. Generally, increasing the number of data points can increase the confidence in the slope of the line. But as few as two data points can be used to determine the slope. In some embodiments, two, three, or four data points are used to determine the slope.

### EXAMPLES

### Example 1

### Reducing False Positives by Measuring Label Intensity at Two Temperatures

PCR reactions were performed to determine whether label intensity at two temperatures can be used to accurately detect the presence of a target nucleic acid while minimizing false positives. The PCR reactions, which were 25 μL each, are described below in Table 1, and the primer sequences used are described in Table 2.

TABLE 1
PCR Reactions

| | | | Final | |
|---|---|---|---|---|
| Component | Volume | | Concentration | |
| 5X Platinum ® Tfi | 5 | μl | 1X | |
| Reaction Buffer | | | | |
| 10 mM dNTP mix, PCR | 0.5 | μl | 200 | μM each |
| grade | | | | |
| 50 mM MgCl2 | 1.5 | μl | 3 | mM |
| Primer mix (10 μM each) | 1 | μl | 0.2 | μM each |
| Template DNA | ≧1 | μl as required | | |
| Platinum ® Tfi Exo(−) | 0.2-0.4 | μl | 2-4 | units |
| DNA Polymerase | | | | |
| Autoclaved distilled water | to 25 | μl | | |

TABLE 2
(Sequences are 5′ to 3′)-Primers

| | |
|---|---|
| TGCGTTCTGACTGAACAGCTGATCGAG | BG_Limiting |
| | Primer |
| TCCTCTTGAAATTCCCGAATGG | BG_Excess |
| | Primer |
| Fam-CTCGAGAAAGGTTGTCGTAAAAC | BG_beacon |
| GCCTCGAG-Dabcyl | |

Four sets of duplicate reactions were set up containing the following amounts of synthetic bacillus globigii (BG) template: 0, 60, 6000, or 600,000 genome copy equivalents. The reactions were cycled 55 times from between 85° C. and 95° C. to between 50° C. and 70° C. The reactions were then reduced to below the Tm of the beacons, between 40° C. and 60° C. Fluorescent reads were taken every 15 seconds for about 5 minutes. The reactions were then heated to between 80° C. and 95° C. and five 15 second reads were taken. The ratios of the fluorescence at 40° C. to 60° C. divided by the fluorescence at 80° C. to 95° C. are shown below in Table 3.

TABLE 3

| BG Template | Fluorescent | % Increase in ratios |
|---|---|---|
| Copies | Ratios | over 0 copies |
| 0 | 0.35 | 0% |
| 60 | 1.36 | 285% |
| 6000 | 2.36 | 569% |
| 600,000 | 2.75 | 680% |

A fluorescent ratio below 1 indicates no target nucleic acid was included in the reaction, while a fluorescent ratio above 1 indicates target nucleic acid was included, as described above. Thus, the results demonstrate that at least as few as 60 copies of plague can be detected reliably using the ratios described herein.

### Example 2

### Reducing False Positives by Measuring Label Intensity Over Time

PCR reactions were performed to determine whether the label intensity as a function of time could be used to determine the presence of a target nucleic acid. PCR reactions were performed, as described above in Example 1.

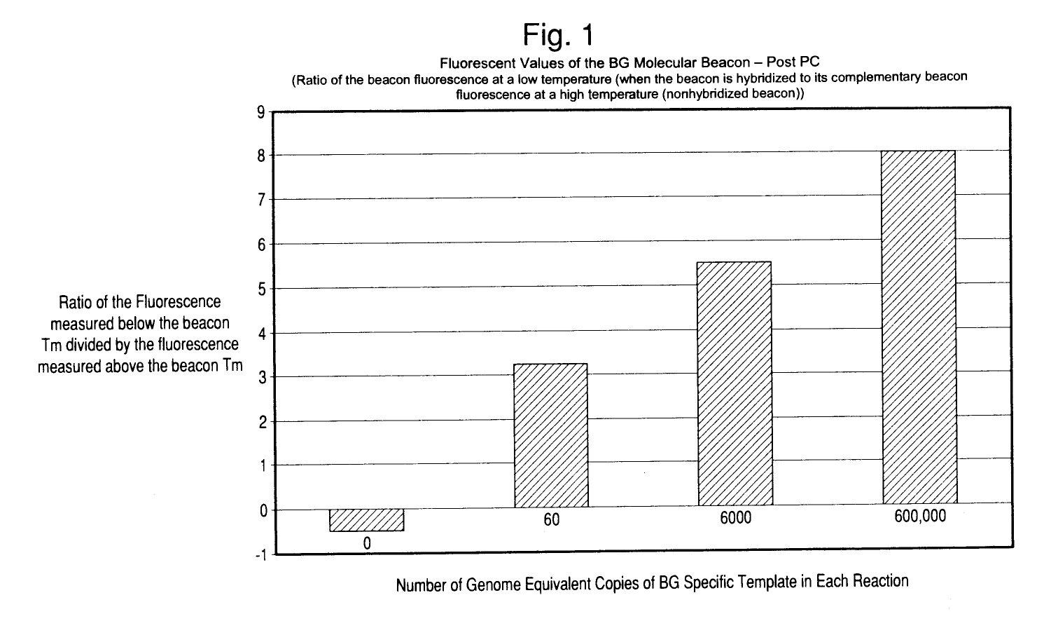

The ratio of the fluorescent values below the beacon Tm divided by the fluorescence values above the beacon Tm were calculated. FIG. 1 and Table 4 below show the calculated ratios for the PCR reactions. Positive ratios were found only in the samples containing the template, while a negative ratio was found for the negative control.

TABLE 4

| BG Template | Fluorescent |
|---|---|
| Copies | Ratios |
| 0 | −0.5 |
| 60 | 3.25 |
| 6000 | 5.5 |
| 600,000 | 8 |

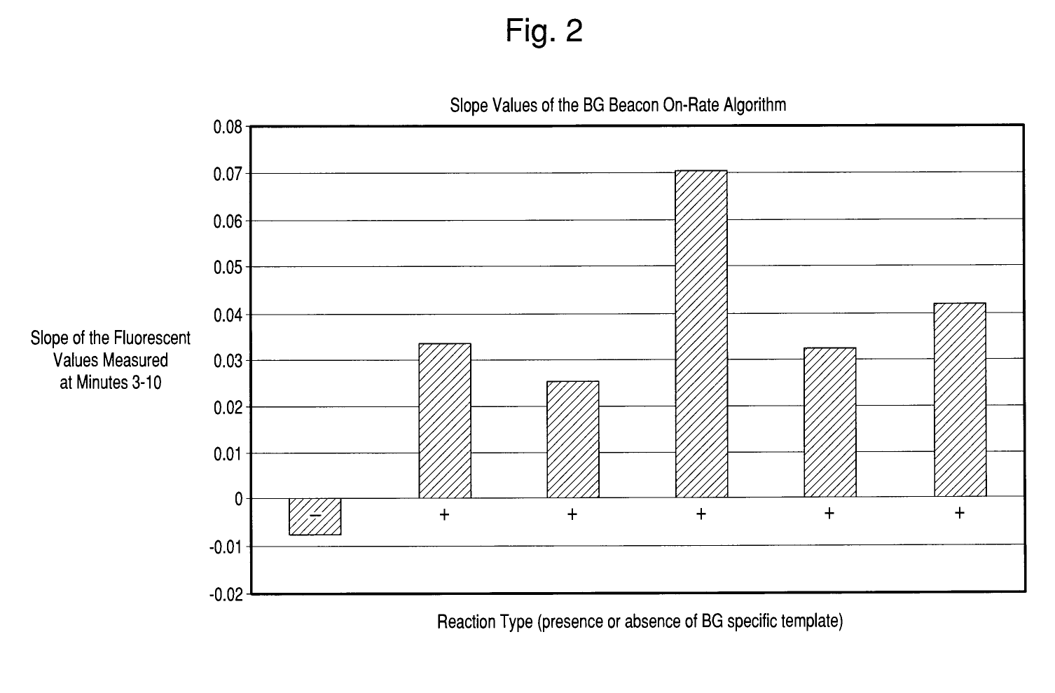

Label intensity was also measured using powder samples that did or did not contain BG template. Specifically, six white powder samples were run on the Bioseeq PLUS instrument, which is available from Smiths Detection, using Training Assay consumables. One sample received negative powder (sample labeled − in FIG. 2), and the remaining five samples received positive BG powder (samples labeled + in FIG. 2). The samples were cycled 55 times from between 85° C. and 95° C. to between 50° C. and 70° C. The reaction temperatures were then reduced to below the Tm of the beacons, between 40° C. and 60° C., and fluorescent reads were taken every 15 seconds for 10 minutes. The fluorescent readings taken from minutes 3 to 10 were plotted on a scatter plot and the slope was calculated by fitting the data using linear regression. This slope value is shown in FIG. 2.

This example demonstrates that measuring label intensity over time can effectively be used to determine whether a target sequence is present. No false positives were found.

### Example 3

### Reducing False Positives by Measuring Label Intensity Over Time and at Different Temperatures with the Anthrax pX01 Assay

PCR reactions were performed to determine whether label intensity at two temperatures at two different times can be used to accurately detect the presence of a target nucleic acid while minimizing false positives. The PCR reactions, which were 25 μL each, are described above in Table 1, and the primer sequences used are described below in Table 5.

TABLE 5
(Sequences are 5′to 3′)-Primers

| | |
|---|---|
| TGGCTAATCAGCTTAAGGAACATCCCACAGAC | Anthrax |
| | pX01_Limiting |
| | Primer |
| TGCATAAAGCTGTAAAACATCACGA | Anthrax |
| | pX01_Excess |
| | Primer |
| CAL 610-CAACGTGGAACAAAATAGCAATGA | Anthrax |
| GGTAACGTTG-Dabcyl | pX01_beacon |

FIG. 2 shows the results of the measurements. Specifically, the values in the target rate column illustrate the on-rate slope and are examples of the differential seen between a positive (POS) and a negative (NEG) call. The three samples that contained no Anthrax template (modules 1,2 and 3) gave NEG calls. The three samples that contained 60,000 anthrax genome copy equivalents (modules 4, 5, and 6) gave POS calls. The NEG calls show a negative slope while the POS calls show a positive slope. The slope was calculated using the 2nd and 4th data point shown in the above plots using the slope function in EXCEL, which is b=Sum((x−Avg(x))*(y−Avg(y)))/Sum((x−Avg(x))̂2) where x is the field in slope that contains the x coordinates and y is the field in slope that contains the y coordinates. The results demonstrate that label intensity at two temperatures at two different times can be used to accurately detect the presence of a target nucleic acid while minimizing false positives.

### Example 4

### Reducing False Positives by Measuring Label Intensity Over Time and at Different Temperatures with the Tularemia Assay

PCR reactions were performed to determine whether label intensity at two temperatures at two different times can be used to accurately detect the presence of a target nucleic acid while minimizing false positives. The PCR reactions, which were 25 μL each, are described below in Table 1, and the primer sequences used are described in Table 6.

TABLE 6
(Sequences are 5′to 3′)-Primers

| | |
|---|---|
| AGCGTAAGATTACAATGGCAGGCTCCAGA | Tularemia_Limiting |
| | Primer |
| GCCCAAGTTTTATCGTTCTTCTCA | Tularemia_Excess |
| | Primer |
| CAL 610-CCTCGTAAGTGCCATGATACA | Tularemia_beacon |
| AGCCGAGG-Dabcyl | |

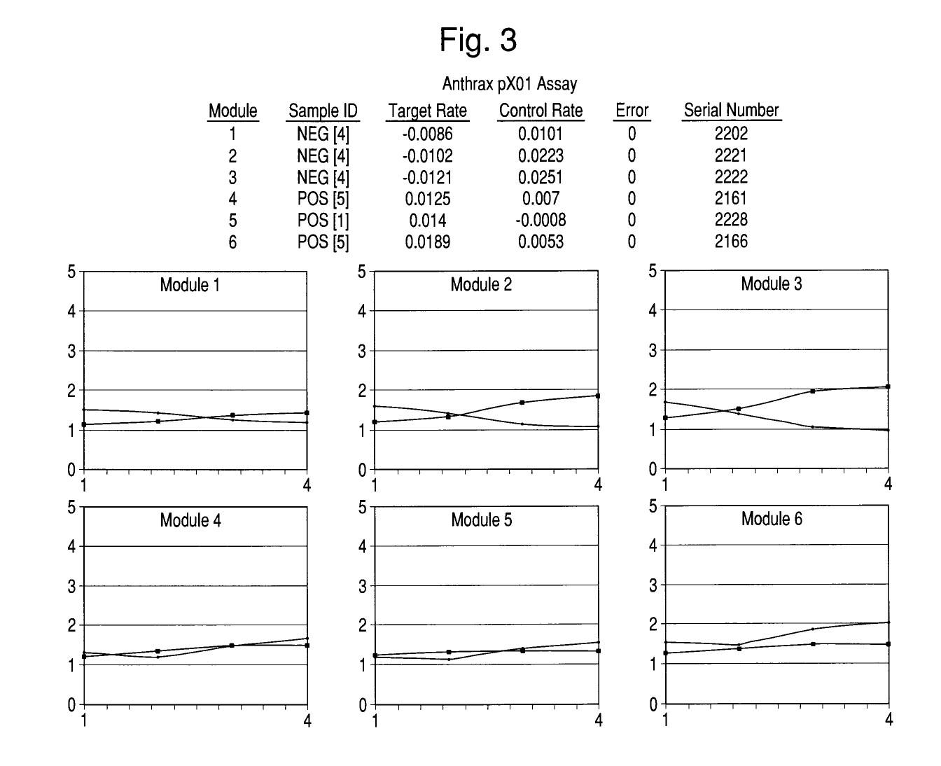

FIG. 3 shows the results of the measurements. Specifically, the values in the target rate column illustrate the on-rate slope and are examples of the differential seen between a positive(POS) and a negative(NEG) call. The three samples that contained no Tularemia template (modules 1, 2, and 3) gave NEG calls. The three samples that contained ˜60,000 Tularemia genome copy equivalents (modules 4, 5, and 6) gave POS calls. Two of the 3 NEG calls show a negative slope while the third NEG call shows a weak positive slope that is below the threshold used for a POS call, which in this case was 0.002. This is an illustration of the background noise of the assay. POS calls show a positive slope. The slope was calculated using the 2nd and 4th data point shown in the above plots using the slope function in EXCEL, which is b=Sum((x−Avg(x))*(y−Avg(y)))/Sum((x−Avg(x))̂2) where x is the field in slope which contains the x coordinates and y is the field in slope which contains the y coordinates. The results demonstrate that label intensity at two temperatures at two different times can be used to accurately detect the presence of a target nucleic acid while minimizing false positives.

1. A method for detecting hybridization of a labeled nucleic acid probe to its target nucleic acid comprising:
(a) contacting a sample suspected of containing a target nucleic acid with a labeled nucleic acid probe that hybridizes with the target nucleic acid;
(b) measuring the label intensity at a first temperature and at a second temperature, wherein the first temperature is less than the second temperature;
(c) calculating the ratio of (i) the label intensity at the first temperature to (ii) the label intensity at the second temperature, wherein a ratio of at least 0.8 indicates the presence of the target nucleic acid.

2. The method of claim 1, wherein a ratio of at least 0.9 indicates the presence of the target nucleic acid.

3. The method of claim 1, wherein the first temperature is below the Tm of the labeled nucleic acid probe and the second temperature is above the Tm of the labeled nucleic acid probe.

4. The method of claim 3, wherein said second temperature is about 85° C., about 86° C., about 87° C., about 88° C., about 89° C., about 90° C., about 91° C., about 92° C., about 93° C., about 94° C., or about 95° C.

5. The method of claim 3, wherein said first temperature is about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., or about 72° C.

6. The method of claim 3, wherein said first temperature is about 50° C. and said second temperature is about 95° C.

7. The method of claim 1, wherein said labeled nucleic acid probe comprises a fluorescent label.

8. The method of claim 7, wherein said nucleic acid probe further comprises a quencher molecule that absorbs the emission of the fluorescent label such that when the quencher molecule and fluorescent label are in close proximity, the fluorescent emission of the fluorescent label is undetectable or at least less than when the quencher molecule and fluorescent label are not in close proximity.

9. The method of claim 1, wherein said nucleic acid probe is a molecular beacon or a linear probe.

10. The method of claim 1, wherein the target nucleic acid is DNA.

11. The method of claim 1, wherein the target nucleic acid is RNA.

12. The method of claim 1, wherein (b) and (c) are repeated at least twice.

13. The method of claim 12, wherein an average ratio is calculated based on the repeated measurements.

14. (canceled)

15. The method of claim 13, wherein the measuring is done following a PCR reaction.

16. The method of claim 13, wherein the PCR reaction comprises:
i) contacting a sample suspected of containing the target nucleic acid with the labeled nucleic acid probe in a solution comprising suitable primers, enzymes and substrates to form a reaction mixture; and
ii) cycling said reaction mixture at denaturing, annealing and extension temperatures suitable for amplification of the target nucleic acid.

17. A method for detecting hybridization of a labeled nucleic acid probe to its target nucleic acid comprising:
(a) contacting a sample suspected of containing a target nucleic acid with a labeled nucleic acid probe that hybridizes with the target nucleic acid;
(b) measuring the label intensity at least two different points in time; and
(c) calculating the slope of the label intensity as a function of time.

18. The method of claim 17, wherein a positive slope indicates the presence of the target nucleic acid when the intensity of the signal generated by the labeled nucleic acid probe is greater when bound to the target nucleic acid as compared to the intensity of the signal generated by the labeled nucleic acid probe when it is not bound to the target nucleic acid.

19. (canceled)

20. The method of claim 17, wherein the target nucleic acid is DNA.

21. The method of claim 17, wherein the target nucleic acid is RNA.

22. The method of claim 17, wherein the labeled nucleic acid probe is a fluorescently labeled nucleic acid probe.

23. The method of claim 22, wherein said nucleic acid probe is a molecular beacon or a linear probe.

24. The method of claim 17, wherein the measuring is done during isothermal conditions.

25. The method of claim 17, wherein the measuring is done following a PCR reaction.

26. The method of claim 25, wherein the measuring is completed within the time period of about 1 to about 10 minutes following the completion of the PCR reaction.

27. (canceled)

28. The method of claim 25, wherein the PCR reaction comprises:
(i) contacting a sample suspected of containing the target nucleic acid with the labeled nucleic acid probe in a solution comprising suitable primers, enzymes, substrates and buffer to form a reaction mixture; and
(ii) cycling said reaction mixture at denaturing, annealing and extension temperatures suitable for amplification of the target nucleic acid.

29. The method of claim 17, wherein the slope is calculated by taking the first derivative of the label intensity as a function of time.

30. The method of claim 29, wherein the label intensity as a function of time is calculated by least squares fitting the label intensity measurements as a function of time.

31. The method of claim 17, wherein the slope is calculated by fitting the label intensity data as a function of time to the following equation:
y=mx+b,
wherein y is label intensity, x is time, and m is the slope.

32. The method of claim 17, wherein the slope of a line is calculated using the formula:
$$m = \frac{{y}_{2} - {y}_{1}}{{x}_{2} - {x}_{1}}$$
where m is the slope of the line, (x₁, y₁) and (x₂, y₂) are the at least two different points in time, and x₁≠x₂.

33. The method of claim 17, further comprising calculating the hybridization kinetics of the labeled nucleic acid probe and target nucleic acid based on the slope of label intensity as a function of time.

34. The method of claim 17, wherein there are at least two labeled nucleic acid probes that each hybridize with a different target nucleic acid, and wherein each probe hybridizes at a different temperature.

35. (canceled)
